(19) United States
(12) Patent Application Publication
(10) Pub. No.: US 2013/0311194 A1
(43) Pub. Date: Nov. 21, 2013

(54) MISLEADING MESSAGE DETECTION

(71) Applicant: Hospital Housekeeping Systems, LLC., Austin, TX (US)

(72) Inventors: Steve Jourdan, Austin, TX (US); Joel Schaubert, Austin, TX (US)

(21) Appl. No.: 13/665,508

(22) Filed: Oct. 31, 2012

Related U.S. Application Data
(60) Provisional application No. 61/553,663, filed on Oct. 31, 2011.

(51) Int. Cl. G06Q 50/22; G06Q 10/06

(52) U.S. Cl. CPC G06Q 50/22; G06Q 10/0633
USPC 705/2

(57) ABSTRACT

A method of managing services in a health care facility includes receiving one or more messages associated with one or more actions to be performed in the hospital; placing the one or more messages in a queue, assessing a queued message, in combination with one or more other messages, against one or more misleading message patterns stored in a memory, determining whether the combination of the message and the one or more other messages matches one or more of the misleading message patterns so as to indicate or suggest that the message is misleading, and if one or more of the misleading message patterns indicates or suggests that the message is misleading, removing the message from the queue or modifying the message.

### PRIORITY CLAIM

This application claims priority to U.S. Provisional Application No. 61/553,663 entitled “SYSTEM AND METHODS OF MANAGING SERVICES IN HEALTH CARE FACILITIES” to Jourdan et al., filed Oct. 31, 2011, which are incorporated herein by reference in its entirety.

### BACKGROUND

1. Field

The present disclosure relates generally to the managing and providing services in health care facilities. More particularly, aspects of the present disclosure relate to systems and methods for assigning and directing personnel and managing workflow in health care facilities.

2. Description of the Related Art

Caring for patients in health care facilities requires a substantial amount of medical and non-medical services. In addition to medical staff, many other members of a health care facility's staff provide services to attend to the needs of patients in the facility and to maintain the facility in continuous operation. Such services include housekeeping, environmental services (“EVS”), bed management, facilities management, admission/discharge/transfer administration, security, and human resources.

In a health care facility setting, some services will be designated as “stat”, or urgent. Typically, a stat designation is made for an action that must be taken promptly in order to meet the urgent needs of a patient or to keep the facility operating at full capacity. From the standpoint of an employee, responding to stat requests may be more stressful than ordinary requests because the employee is under more pressure to complete the task quickly. In many cases, a stat request may be associated with an urgent medical situation, such as cardiac arrest or another emergency situation.

In many health care facilities, computer systems are used to help manage services provided to patients. In some health care facilities, requests for some services, such as bed cleaning or patient transport, are triggered by messages from an Admission Discharge Transfer (“ADT”) system. For example, a message from an ADT system that a patient has just been discharged from the room may trigger a bed cleaning request. Some systems may assign requests to staff members in a way that one or more of the staff members becomes overloaded with stat requests.

For a number of reasons, some of the messages sent from an ADT system may be misleading or even false. For example, in Health Level 7 (“HL7”) message feeds obtained from a hospital ADT system, there may be instances in which one message relating to the state of a hospital bed is invalidated by a later message. For instance, messages from the HL7 feed might describe a patient who is transferred from bed A to bed B, then a short time later transferred from bed B to back to bed A. In this case, a patient may have been “transferred” in the ADT system in error, and a second “transfer” made in the ADT system to correct the error. In this situation, a cleaning team may be dispatched to clean a room only to find upon reaching the room that the room is still occupied. There are many such examples of misleading messages, some of which involve multiple messages. Using a system that fails to identify or account for misleading or false messages may result in inefficiencies in the operation of a health care facility, such as wasted labor in responding to incorrect information.

### SUMMARY

Systems and methods for managing and providing services in health care facilities are described. According to one embodiment, a method of managing services in a health care facility includes receiving one or more stat requests; automatically identifying, for at least one of the stat requests, a candidate to perform at least one task associated with the stat request; automatically determining whether the candidate already has a predetermined maximum number of stat requests; if the candidate already has the predetermined maximum number of stat requests, notifying a manager that the candidate is not available to fulfill the stat request; and paging at least one person to fulfill the stat request.

According to one embodiment, a system includes a processor and a memory coupled to the processor and configured to store program instructions executable by the processor to implement a method of managing services in a health care facility that includes receiving one or more stat requests; automatically identifying, for at least one of the stat requests, a candidate to perform at least one task associated with the stat request; automatically determining whether the candidate already has a predetermined maximum number of stat requests; if the candidate already has the predetermined maximum number of stat requests, notifying a manager that the candidate is not available to fulfill the stat request; and paging at least one person to fulfill the stat request.

According to one embodiment, a tangible, computer readable medium includes program instructions are computer-executable to implement a method of managing services in a health care facility that includes receiving one or more stat requests; automatically identifying, for at least one of the stat requests, a candidate to perform at least one task associated with the stat request; automatically determining whether the candidate already has a predetermined maximum number of stat requests; if the candidate already has the predetermined maximum number of stat requests, notifying a manager that the candidate is not available to fulfill the stat request; and paging at least one person to fulfill the stat request.

According to one embodiment, a method of managing bed cleaning services in a health care facility includes receiving one or more cleaning request; determining, for at least one of the cleaning requests, a value of a bed state for one or more beds, wherein the possible values of bed state comprise a dirty state and a stat state; and selecting, for the at least one request, a cleaner from among two or more candidate cleaners, wherein, in selecting the cleaner for the at least one request, a candidate cleaner is automatically excluded from consideration if the candidate cleaner: has been paged for a prior stat request that is not yet completed; or is in progress on a prior stat request; wherein, in selecting the cleaner for the request, cleaners who are free have priority over cleaners who have been paged for a prior non-stat request that is not yet completed; and cleaners who are in progress on a prior non-stat request.

According to one embodiment, a system includes a processor and a memory coupled to the processor and configured to store program instructions executable by the processor to implement a method of managing bed cleaning services in a health care facility that includes receiving one or more cleaning request; determining, for at least one of the cleaning requests, a value of a bed state for one or more beds, wherein the possible values of bed state comprise a dirty state and a stat state; and selecting, for the at least one request, a cleaner from among two or more candidate cleaners, wherein, in selecting the cleaner for the at least one request, a candidate cleaner is automatically excluded from consideration if the candidate cleaner: has been paged for a prior stat request that is not yet completed; or is in progress on a prior stat request; wherein, in selecting the cleaner for the request, cleaners who are free have priority over cleaners who have been paged for a prior non-stat request that is not yet completed; and cleaners who are in progress on a prior non-stat request.

According to one embodiment, a tangible, computer readable medium includes program instructions are computer-executable to implement a method of managing bed cleaning services in a health care facility that includes receiving one or more cleaning request; determining, for at least one of the cleaning requests, a value of a bed state for one or more beds, wherein the possible values of bed state comprise a dirty state and a stat state; and selecting, for the at least one request, a cleaner from among two or more candidate cleaners, wherein, in selecting the cleaner for the at least one request, a candidate cleaner is automatically excluded from consideration if the candidate cleaner: has been paged for a prior stat request that is not yet completed; or is in progress on a prior stat request; wherein, in selecting the cleaner for the request, cleaners who are free have priority over cleaners who have been paged for a prior non-stat request that is not yet completed; and cleaners who are in progress on a prior non-stat request.

According to one embodiment, a method of assigning tasks to members of an organization includes receiving a selection of an assignment setting for each of at least one of one or more groups of members of the organization, wherein the assignment setting selection at least partially determines how tasks are to be assigned to members of the at least one group; receiving one or more requests for a task to be performed by one or more of the members of one or more of the groups; and assigning at least one of the requests to at least one of the members of at least one of the groups based at least in part on the assignment setting for the member's group.

According to one embodiment, a system includes a processor and a memory coupled to the processor and configured to store program instructions executable by the processor to implement a method of assigning tasks to members of an organization that includes receiving a selection of an assignment setting for each of at least one of one or more groups of members of the organization, wherein the assignment setting selection at least partially determines how tasks are to be assigned to members of the at least one group; receiving one or more requests for a task to be performed by one or more of the members of one or more of the groups; and assigning at least one of the requests to at least one of the members of at least one of the groups based at least in part on the assignment setting for the member's group.

According to one embodiment, a tangible, computer readable medium includes program instructions are computer-executable to implement a method of assigning tasks to members of an organization that includes receiving a selection of an assignment setting for each of at least one of one or more groups of members of the organization, wherein the assignment setting selection at least partially determines how tasks are to be assigned to members of the at least one group; receiving one or more requests for a task to be performed by one or more of the members of one or more of the groups; and assigning at least one of the requests to at least one of the members of at least one of the groups based at least in part on the assignment setting for the member's group.

According to one embodiment, a portable electronic device includes a portable housing; a processor coupled to the portable housing; an input device coupled to the processor; a display coupled to the processor, wherein the display is configured to simultaneously display to a user: a request panel comprising a list of one or more cleaning requests; and a cleaner panel comprising a list of one or more cleaners.

According to one embodiment, a method of managing services in a health care facility includes receiving one or more messages associated with one or more actions to be performed in the hospital; placing the one or more messages in a queue; assessing a queued message, in combination with one or more other messages, against one or more misleading message patterns stored in a memory; determining whether the combination of the message and the one or more other messages matches one or more of the misleading message patterns so as to indicate or suggest that the message is misleading; and if one or more of the misleading message patterns indicates or suggests that the message is misleading, removing the message from the queue or modifying the message.

According to one embodiment, a system includes a processor and a memory coupled to the processor and configured to store program instructions executable by the processor to implement a method of managing services in a health care facility that includes receiving one or more messages associated with one or more actions to be performed in the hospital; placing the one or more messages in a queue; assessing a queued message, in combination with one or more other messages, against one or more misleading message patterns stored in a memory; determining whether the combination of the message and the one or more other messages matches one or more of the misleading message patterns so as to indicate or suggest that the message is misleading; and if one or more of the misleading message patterns indicates or suggests that the message is misleading, removing the message from the queue or modifying the message.

According to one embodiment, a tangible, computer readable medium includes program instructions are computer-executable to implement a method of managing services in a health care facility that includes receiving one or more messages associated with one or more actions to be performed in the hospital; placing the one or more messages in a queue; assessing a queued message, in combination with one or more other messages, against one or more misleading message patterns stored in a memory; determining whether the combination of the message and the one or more other messages matches one or more of the misleading message patterns so as to indicate or suggest that the message is misleading; and if one or more of the misleading message patterns indicates or suggests that the message is misleading, removing the message from the queue or modifying the message.

According to one embodiment, a method of managing services includes receiving one or more messages associated with one or more actions to be performed; placing the one or more messages in a queue; assessing whether or not at least one received message is a type of message to be held; if the received message is a type of message to be held, applying a hold to the message; unholding the message after a period of time has elapsed; assessing the unheld message, in combination with one or more other messages, against one or more misleading message patterns stored in a memory; determining whether the combination of the unheld message and the one or more other messages matches one or more of the misleading message patterns so as to indicate or suggest that the message is misleading; and if one or more of the misleading message patterns indicates or suggests that the message is misleading, removing the message from the queue or modifying the message.

According to one embodiment, a system includes a processor and a memory coupled to the processor and configured to store program instructions executable by the processor to implement a method of managing services that includes receiving one or more messages associated with one or more actions to be performed; placing the one or more messages in a queue; assessing whether or not at least one received message is a type of message to be held; if the received message is a type of message to be held, applying a hold to the message; unholding the message after a period of time has elapsed; assessing the unheld message, in combination with one or more other messages, against one or more misleading message patterns stored in a memory; determining whether the combination of the unheld message and the one or more other messages matches one or more of the misleading message patterns so as to indicate or suggest that the message is misleading; and if one or more of the misleading message patterns indicates or suggests that the message is misleading, removing the message from the queue or modifying the message.

According to one embodiment, a tangible, computer readable medium includes program instructions are computer-executable to implement method of managing services that includes receiving one or more messages associated with one or more actions to be performed; placing the one or more messages in a queue; assessing whether or not at least one received message is a type of message to be held; if the received message is a type of message to be held, applying a hold to the message; unholding the message after a period of time has elapsed; assessing the unheld message, in combination with one or more other messages, against one or more misleading message patterns stored in a memory; determining whether the combination of the unheld message and the one or more other messages matches one or more of the misleading message patterns so as to indicate or suggest that the message is misleading; and if one or more of the misleading message patterns indicates or suggests that the message is misleading, removing the message from the queue or modifying the message.

### BRIEF DESCRIPTION OF THE DRAWINGS

FIG. 1 illustrates one embodiment of a health care services system including management systems connected to user devices over a network.

FIG. 2 illustrates one embodiment of a system including a hospital with a network connection to a cloud computing system.

FIG. 3 illustrates one embodiment of a system of health care facilities connected over a network to a cloud computing system.

FIG. 4 illustrates one embodiment of an electronic bed board.

FIG. 5 illustrates one embodiment of a user interface for managing environmental services.

FIG. 6 illustrates one embodiment of a user interface for an EVS manager dispatch.

FIG. 7 illustrates one embodiment of managing services that includes a limit on stat requests.

FIG. 8 illustrates one embodiment of a truth table for making bed cleaning assignments.

FIG. 9 is a flow diagram illustrating a cleaner page initiated based on a message that a bed has gone dirty.

FIG. 10 is a flow diagram illustrating paging based on a message that a cleaner has become available.

FIG. 11 illustrates one embodiment of making assignments based on a combination of values of bed state and cleaner state.

FIG. 12 illustrates one embodiment of managing cleaning services that excludes cleaners with uncompleted stat requests.

FIG. 13 illustrates one embodiment of assigning tasks based on a group assignment setting.

FIG. 14 illustrates one embodiment of managing services in a health care facility that includes detection of misleading messages.

FIG. 15 illustrates one embodiment of managing services that includes holding and evaluating messages against misleading message patterns.

FIG. 16 is a flow diagram illustrating one embodiment of a delay pipeline for responding to service requests.

While the invention is described herein by way of example for several embodiments and illustrative drawings, those skilled in the art will recognize that the invention is not limited to the embodiments or drawings described. It should be understood, that the drawings and detailed description thereto are not intended to limit the invention to the particular form disclosed, but on the contrary, the intention is to cover all modifications, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION OF EMBODIMENTS

As used herein, an “action” means an act, task, milestone, operation, step, process, or communication that can be performed, completed, or achieved by one or more persons.

As used herein, “call message” means a message that requests, directs, or commands an action to be carried out by one or more persons, or indicates a need for an action by one or more persons.

As used herein, “candidate” means a person who may be considered for a task.

A “communications network” refers to a system including one or more communication channels (i.e., lines, satellite frequency, radio waves, etc.) interconnecting one or more nodes (i.e., servers, routers, computers, communication devices, etc.). Examples of a “communications network” include a cellular telephone network, the internet, a local area network (LAN), or a wide area network (WAN).

As used herein, “fictional” message means a message that is initiated or entered into a system by user even though the message is known to not reflect actual conditions or needed actions. A fictional message may be entered for administrative purposes, such as for billing, operations, or logistics.

As used herein, “health care facility”, includes any facility in which medical health care services are provided. Examples of a health care facility includes a hospital, a Federally qualified health center, a rural health clinic, a nursing home, personal care home, a home health agency, a hospice program, a public health clinic, a State or local department of public health, a skilled nursing facility, an ambulatory surgical center.

As used herein, an “HL7” message means a message based on an HL7 standard, such as the HL7 version 2.x or HL7 version 3 standard developed by the Health Level Seven community.

As used herein, a “misleading message” is a message that is false, fictional, or otherwise misleading. A misleading message may indicate or suggest that a condition exists (such as a discharge of a patient from a hospital room) when the condition does not in fact exist, or is intended or likely to be reversed or cancelled. A misleading message may also indicate or suggest that an action (such as a bed cleaning) should be undertaken when no such action is or will be required. In some embodiments, a misleading message may indicate that a bed is occupied when it is in fact not occupied, or, conversely, that a bed is not occupied when it is in fact occupied. In certain embodiments, a misleading message is the result of bed hiding. A misleading message may include an ephemeral or fleeting message that is temporarily true, but with the intention, expectation, or likelihood of being reversed, cancelled, or superseded.

As used herein, “misleading message pattern” means a pattern in a set or group of two or more messages that indicates or suggests that at least one message in the set or group of messages is a misleading message.

As used herein, “occupant” means a person who is occupying a room or area. An occupant may be a patient in a hospital room.

As used herein, to “page” means to send a message to one or more persons to notify the person of one or more actions.

As used herein, “request” means a request to do a task, or an indication that a task should be performed. For example, a cleaning request may be a message indicating that one or more beds are dirty and need to be cleaned.

As used herein, “task” includes a specific task, set of tasks, a shift, or duties.

As used herein, “stat” means urgent or rush.

As used herein, “suspect” message means a message that has at least one characteristic making it appear suspicious, or indicating or suggesting that it could be misleading.

As used herein, “unhold” means to release a message from a hold condition.

### Systems for Managing Services at Health Care Facilities

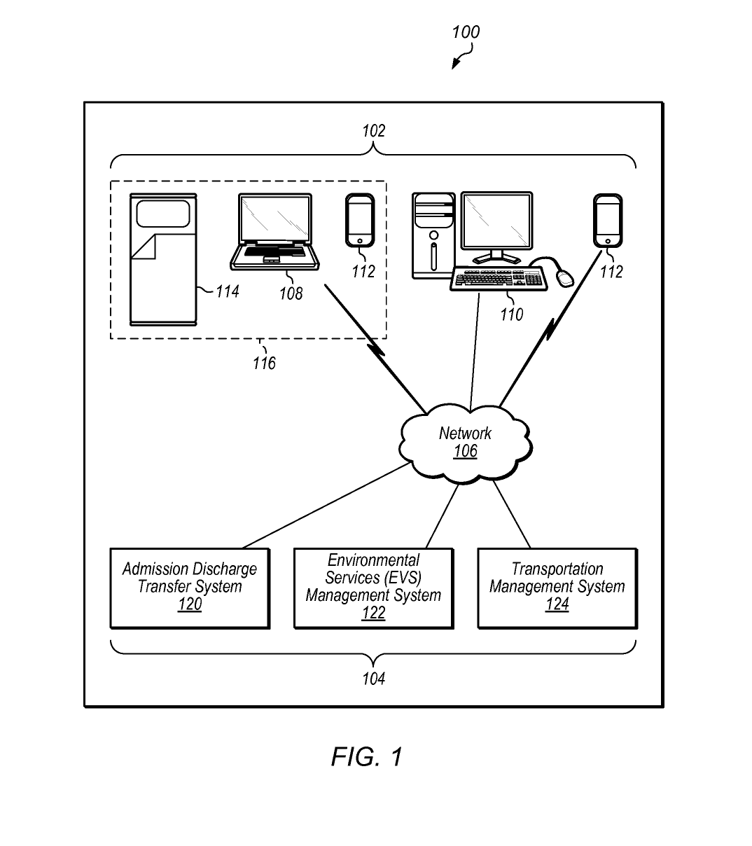

In various embodiments, a system for managing services in a health care facility includes user devices connected to management systems over a network. FIG. 1 illustrates one embodiment of a health care services system including management systems connected to user devices over a network.

System 100 includes user devices 102, management systems 104, and network 106. User devices 102 may be connected with one another and with management systems 104 over network 106. Management systems 104 may be connected with one another by way of network 106. In various embodiments, system 100 may be used to manage and provide services in a health care facility, such as a hospital.

User devices 102 include notebook computer 108, desktop computer 110, and portable electronic devices 112. Examples of portable electronic devices include a smart phone, a tablet computer, or a pager. User devices 102 may enable communication, task management, and reporting between one another and between other systems on network 106.

User devices 102 may be connected to the network over any suitable medium, such as electrical or optical cable, or via any suitable wireless standard such as IEEE 802.11 (“Wi-Fi”), IEEE 802.16 (“WiMax”), or cellular network.

In some embodiments, user devices connected via a network are operated from patient rooms in a health care facility. For example, as illustrated in FIG. 1, notebook computer 108 may be located in room 116. Notebook computer 108 may be operated in connection with management of bed 114.

Management systems 104 include admission/discharge/transfer (“ADT”) system 120, environmental services (“EVS”) management system 122, and transportation management system 124. ADT system 120 may be used to manage patient admissions, discharges, and transfers in a health care facility, such as admission, discharge, and transfer of a patient in room 116.

EVS management system 122 may be operated to manage environmental services in a facility. Examples of environmental services may include bed cleaning, room cleaning, housekeeping, disinfection, decontamination, room supplies, or laundry. In some embodiments, EVS management system 122 receives and responds to messages sent from ADT system 120. For example, EVS management system 122 may generate cleaning request for a room in response to receiving a patient discharge notice from ADT system 120. The discharge message may be sent over network 106.

In FIG. 1, ADT system 120, EVS management system 122, and transportation management system 124 are depicted as a separate systems. Any or all of these systems may, however, be combined in some embodiments into one computer system, such as a management server. In one embodiment, ADT system 120 is provided on one computer system and EVS management system 122 and transportation management system 124 are provided on a second computer system. In some embodiments, EVS management system 122 and transportation management system 124 receive HL7 messages issued from ADT system 120.

Transportation system 124 may be operated to manage transportation services at a facility. Examples of transportation services may include transporting a patient from one location to another, transporting equipment from one location to another, or transporting items for medical procedures or testing. A patient or resident of a facility may be transported into a facility to another location, from another location to the facility, or from one location to another within a facility, such as from one room to another room in a hospital. Equipment may include systems for providing medical treatment or diagnosis, such as an x-ray machine, ultrasound machine, surgical table, or treadmill. Medical items may include medicine, plasma, blood samples, or transplant organs.

Although for illustrative purposes only a single notebook computer and a single desktop computer are shown in FIG. 1, a system may in various embodiments have any number, and any of various types, of portable or fixed electronic devices.

For illustrative purposes, FIG. 1 shows user devices including a notebook, smart phone, and desktop workstation. Nevertheless, in various embodiments, other types of user devices may be used by cleaners and other personnel. For example, each cleaner on the staff of a hospital may carry a pager. As another example, a cleaner may access information relating to the cleaner's assignments from a fixed workstation, such as a personal computer workstation in an occupant's room. In certain embodiments, the portable electronic devices connected to a system include tablet computers or smart phones.

In some embodiments, a bed management system is integrated with an ADT system, an EVS management system 122, or both. For example, a bed management system may be integrated with ADT system 120.

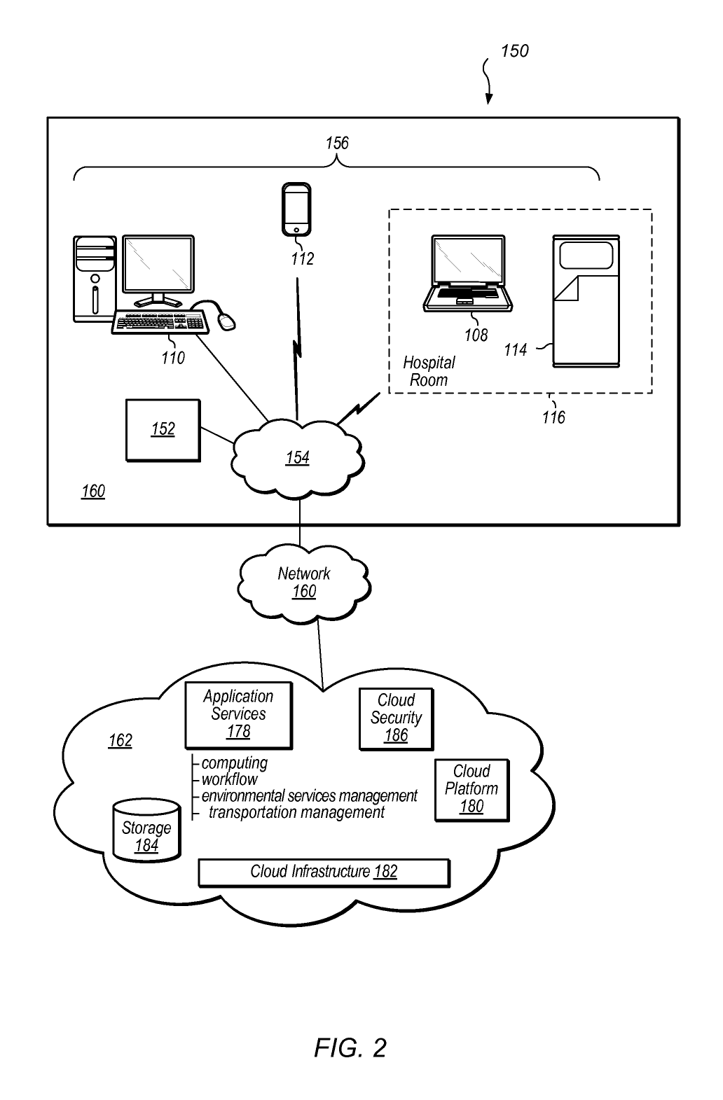

In some embodiments, management of a health care facility includes accessing a cloud computing system over a communications network. FIG. 2 illustrates one embodiment of a system including a hospital with a network connection to a cloud computing system. System 150 includes hospital computing system 152, network 154, and user devices 156. Hospital computing system 152 and user devices 156 may be connected by way of network 154. Hospital computing system 152, network 154, and user devices 156 may be located at site 160. Hospital computing system 152 and user devices 156 may be connected to cloud computing system 162 by way of network 160. Hospital personnel may operate user devices 156 to access hospital computing system 152 and cloud computing system 162.

Networks 154 and 160 may include any suitable data network or combination of networks that enable the exchange of information between electronic systems. For example, networks 160 may include one or more Local Area Networks (LANs) such as Ethernet networks, as well as Wide Area Networks (WANs), Metropolitan Area

Networks (MANs), or other data or telecommunication networks implemented over any suitable medium, such as electrical or optical cable, or via any suitable wireless standard such as IEEE 802.11 (“Wi-Fi”), IEEE 802.16 (“WiMax”), etc. In various embodiments, all or a portion of networks 160 may include the network infrastructure commonly referred to as the Internet. In other embodiments, networks 154 and 160 may be entirely contained within an enterprise and not directly accessible from the Internet.

Cloud computing system 162 may provide remote computing resources, remote storage resources, or both, for systems connected to cloud computing systems 162. For example, cloud computing system 162 may provide cloud computing services to personnel at site 160.

Various system architectures may be employed in cloud computing system 162. Systems and components of cloud computing system 162 may be at a single physical location, such as a data center, or distributed among any number of locations. Cloud computing system 162 includes cloud application services 178, cloud platform 180, cloud infrastructure 182, cloud data storage 184, and cloud security 186. Examples of application services 178 include computing services, remote data storage services, workflow management, production control, content management, accounting, administration, human resources, and enterprise resource planning. Cloud application services 178 may access cloud data storage 184.

In some embodiments, some of health facilities services are managed using application services in a computing cloud. In one embodiment, environmental services, transportation services, or both, are performed as one of application services 178. In certain embodiments, environmental services or transportation services in a cloud receive a message feed from a local computing system, such as one or more of systems 204 at sites 202. In one embodiment, system 204 includes an ADT system that transmits HL7 messages to application services in cloud computing system 162.

Cloud infrastructure 182 may encompass a variety of physical resources, such as computing devices, servers, block storage, mass storage devices, file servers, software, and network systems. In some embodiments, a cloud computing system encompasses virtualized resources, such as virtualized data storage or virtualized hardware.

In some embodiments, a service provider provides resources to customers by way of cloud computing resources. In some embodiments, computation resources are rented or leased to customers of the service provider. In certain embodiments, services are provided to users at sites as software as a service (“SaaS”) or platform as a service (“Paas”). Services may be provided to each customer on an on-demand basis.

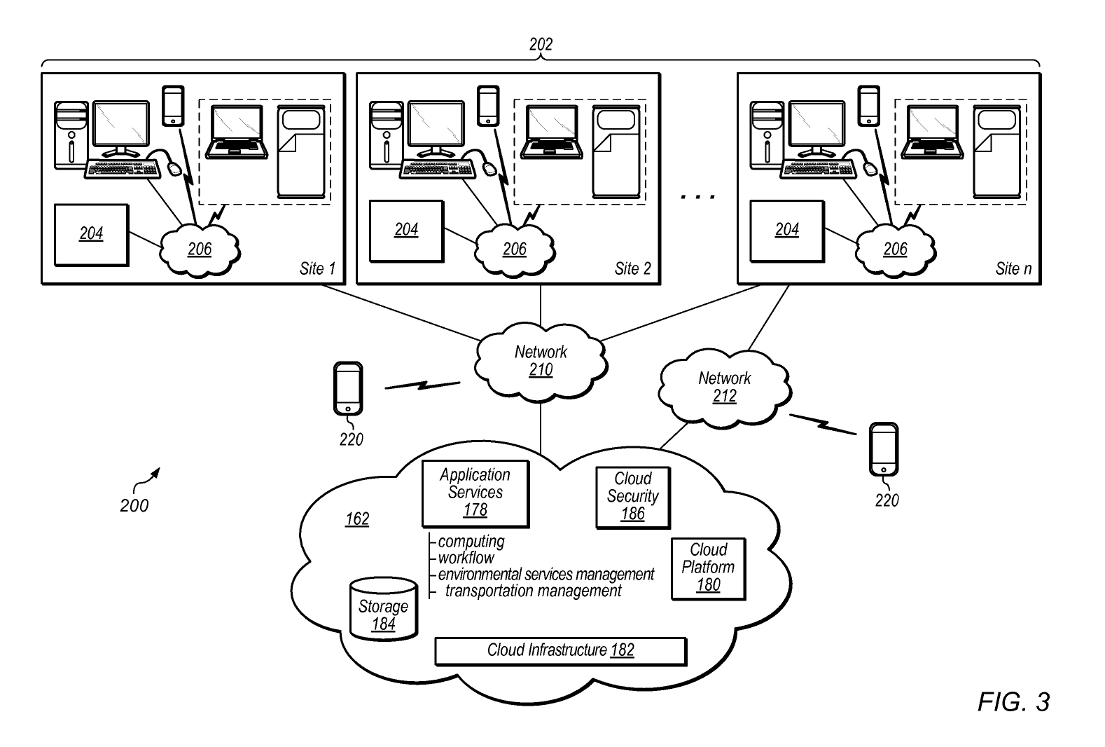

FIG. 3 illustrates one embodiment of a system of health care facilities connected over a network to a cloud computing system. System 200 includes health care sites 202 includes site 1 through site n. Each of sites 202 may include a site computing system 204. Site computing systems 204 may include, for example, a network of computing devices distributed at the site and connected to one another by way of network 206. Each of site computing systems 204 may be connected to cloud computing system 162 by way of network 210. Cloud computing system 162 may be similar to the cloud computing system described above relative to FIG. 2. In certain embodiments, site computing systems 204 may be connected to one another by way of network 210.

In various embodiments, some of sites may be connected over a different network than other sites. For example, as shown in FIG. 3, Site n may be connected to cloud computing system 162 over network 212. In some embodiments, one or more sites are connected over a private network. For example, in the embodiment shown in FIG. 3, network 210 may be a public network and network 212 may be a private network.

In various embodiments, a user may communicate over systems in system 200 from locations external to sites 202 and cloud computing system 176. For example, users not located at one of sites 202 may communicate with users at sites 202 by way of portable electronic devices 220. Portable electronic devices 220 may be located anywhere, including a manager's, administrator's, or physician's home, a health clinic, or any other location.

Although for illustrative purposes only three sites are shown in FIG. 3, a system may include any number of sites and any number of site computer systems. In some embodiments, one site has two or more site computer systems.

### Bed Board/User Interface

In some embodiments, a system for managing services in a health care facility includes one or more electronic bed boards. Electronic bed boards may be accessed and displayed on one more computer systems in a health care facility. In some embodiments, each user is authenticated (such as by login or access card) before access is to the system is provided.

In some embodiments, a bed board is accessible to a user on a portable electronic device. In certain embodiments, a bed board is accessible to a user by way of a smart phone. Users may review bed board information, make or accept assignments, or enter information relating to specific personnel, cleaning services, rooms or assignments.

In some embodiments, a Summary Bed Board view provides information relating to the status of beds/rooms and their availability. A view may include a combination of two or more of: (1) Number of Clean Beds; (2) Number of Needed Beds; (3) The Sex of Patients Waiting for Beds; (4) Listing of Beds Currently Available; (5) Listing of Beds Available Soon; and (6) Sex Corresponding to the Beds Available and Available Soon.

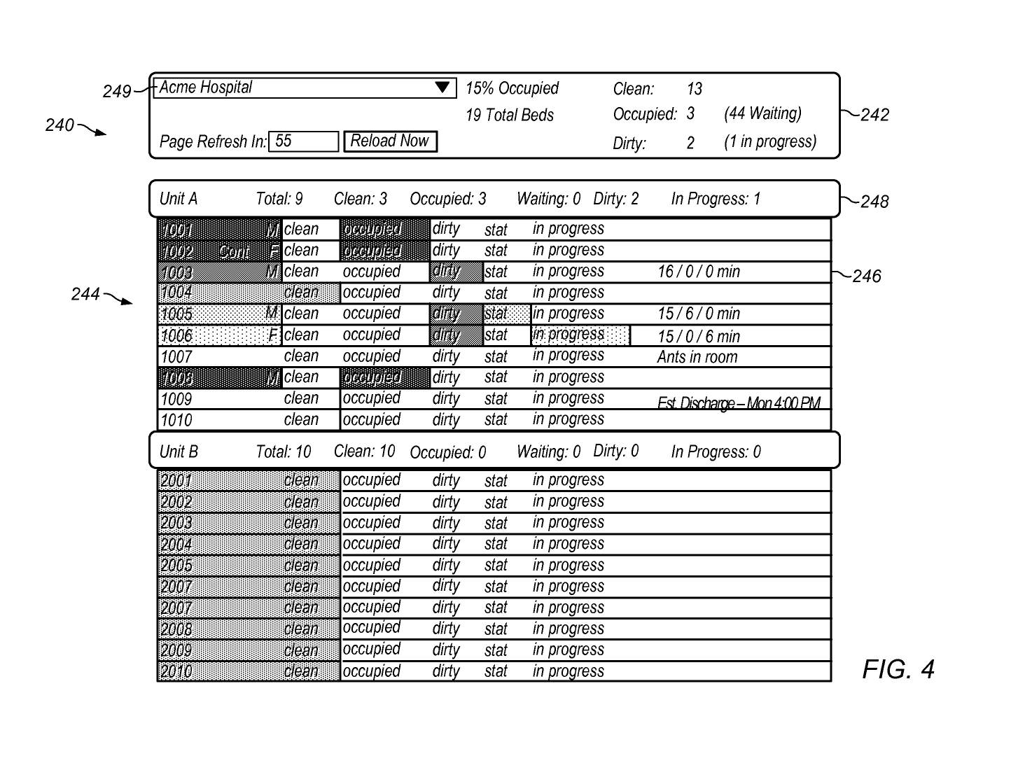

FIG. 4 illustrates one embodiment of an electronic bed board. Electronic bed board 240 includes summary box 242 and unit panels 244. A separate unit panel 244 may be provided for each unit in a facility (for example, a hospital unit). Each of unit panels 244 may include room listing 246 and unit summary 248. Each row of the listing may include information on a bed in the identified room. The following notes reflect examples of information that may be included in a room listing in various embodiments.
Bed 1002. “Cont” may indicate one of 3 generic isolation conditions (contact, droplet, airborne). Isolation condition information may be important to cleaners because they must protect themselves differently for different isolation condition types. An indication that a room is ISO may also appear in a cleaner's pager message
Bed 1005. 15/6/0 may indicate that the room has been dirty for 15 minutes, and was marked as stat 6 minutes ago
Bed 1006. 15/0/6 may indicate that the room has been dirty for 15 minutes, 0—never marked as stat, cleaning started 6 minutes ago.
Bed 1007. A grey color may indicate that the room is not available for use. Comments may indicate nature of the problem (“Ants in room”).
Bed 1008. Hospital staff has noted the likely discharge time at 4 pm Monday.

Summary box 242 includes facility drop down menu 249. Facility drop down menu 249 may allow a user to change between bed boards of different health care facilities.

In some embodiments, a computer system includes a user interface for managing environmental services. In one embodiment, an environmental services bed board includes of a view of one or more of following information: (1) Number of Beds; (2) Number of Clean Beds; (3) Number of Occupied Beds; (4) Number of Dirty Beds; (5) Time Beds Have Been Dirty; (6) Cleaner(s) Assigned; (7) Cleaners Available; (8) Duration of Pending Cleaning Request; (9) Duration of STAT Cleaning. (10) Last Time Bed/Room Cleaned; and (11) Last Cleaner to Clean Bed/Room.

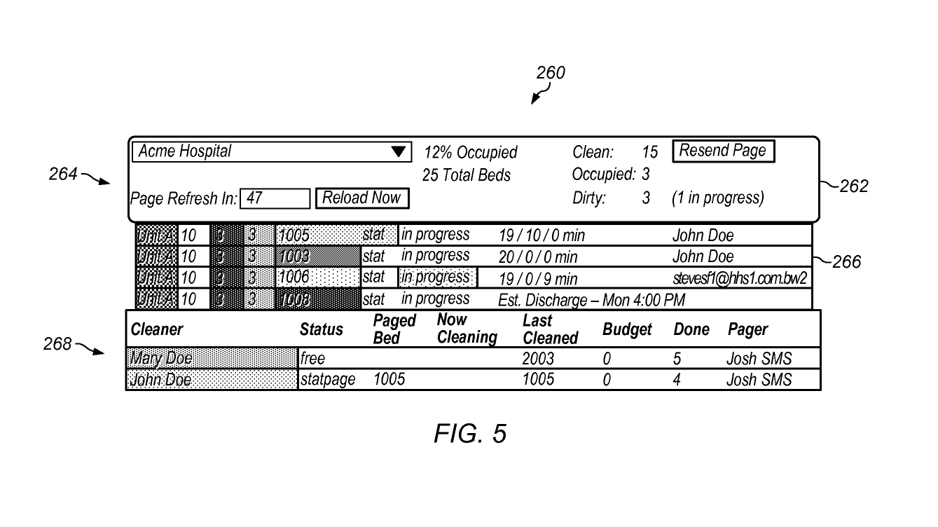

FIG. 5 illustrates one embodiment of a user interface for managing environmental services. EVS Manager 260 includes EVS management panel 262. EVS management panel 262 includes room summary 264, room listing 266, and cleaner listing 268.

In room listing 266, the second, third, fourth columns indicate how many beds, occupied beds, and clean beds are in the same unit as the bed shown on that row. If a bed is in a unit that contains many other clean beds, service for that bed is not likely to be a stat job. On the other hand, if there are no other clean beds in the unit, then service for that bed may be a stat job.

In some embodiments, some or all of the cleaners on a list of cleaners may include a visual indication that shows how busy the cleaner is. For example, a color may be shown on each cleaner's name column to indicate how busy the cleaner is. Each color in a set of colors may indicate a different level of business for a cleaner.

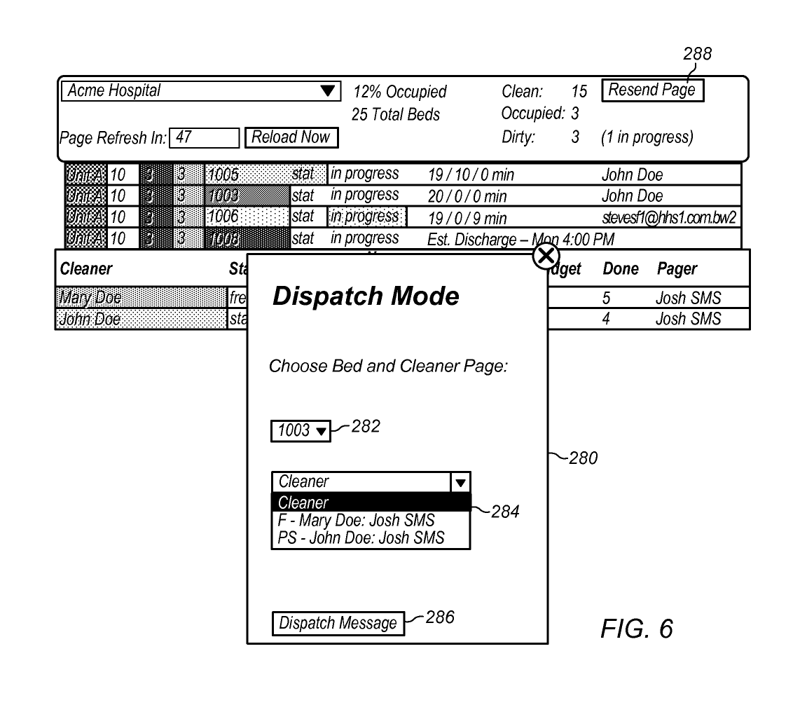

In some embodiments, a manager activates a dispatch window from a dual-pane management window for managing EVS services. FIG. 6 illustrates one embodiment of a user interface for an EVS manager dispatch. Dispatch mode window 280 may be overlaid on an EVS management panel, such as EVS management panel 262 shown in FIG. 5. Dispatch mode window 280 includes bed drop down menu 282, cleaner drop down menu 284, and dispatch button 286.

If a manager clicks resend page 288, the manager can do a manual dispatch. A manual dispatch may include:

selecting a room
selecting a cleaner for the room
dispatching a dispatch message

In some embodiments, an indicator, such as a code, is displayed in association with each cleaner's name that indicates how busy the cleaner is. The listing of cleaners may be sorted based on how busy the cleaners are. In some embodiments, the listing is sorted from least busy to most busy. The manager picks from someone from the top of the list because they are sorted as less busy. An example set of codes is listed below.

F—free
P—paged
I—in progress
PS—paged to a stat room
IS—in progress on a stat room

In progress on a stat may be sorted to the bottom of the listing. In certain embodiments, cleaner status may be indicated using different colors (for example, green for free, red for unavailable).

After selecting a room and a cleaner, the manager may click on dispatch button 286 to automatically generate a dispatch message.

In some embodiments, a user manages services in a health care facility using a portable electronic device. In one embodiment, a portable electronic device includes a portable housing, a processor, and input device (such as a touch screen), and a display. The display may simultaneously display to a user a request panel that includes one or more cleaning requests and a cleaner panel that includes a list of one or more cleaners. The display may be, for example, a two panel display as described above relative to FIG. 5.

### Services Management

In various embodiments, services in a health care facility are managed using one or more computer systems. Examples of services that may be managed using a computer system include environmental services, cleaning, admission/discharge/transfer, bed management, or transportation. In some embodiments, services are managed over a user devices connected to a network, such as a portable electronic device.

In some embodiments, a limit is placed on the number of a stat requests assigned to a staff member of a medical or personal care facility. In one embodiment, stat cleaning requests are distributed in a limited manner to reduce the stress level for environmental services (EVS) staff.

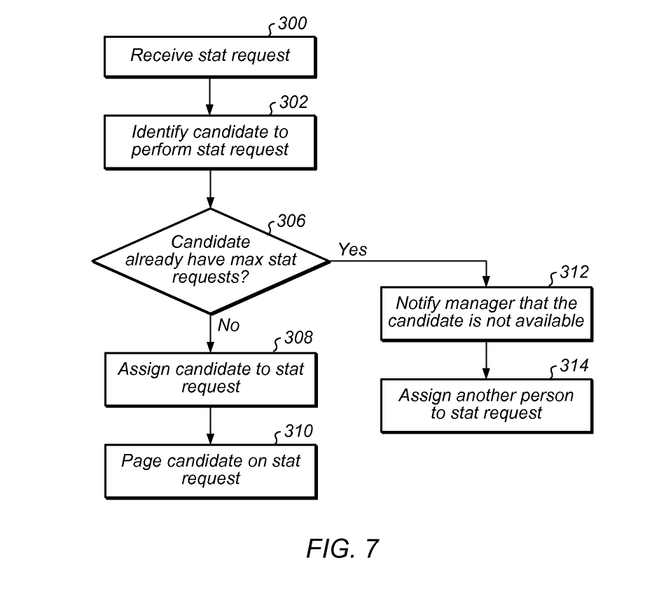

FIG. 7 illustrates one embodiment of managing services that includes a limit on stat requests. At 300, one or more stat requests are received. Stat requests may be, for example, an urgent request to clean a bed or a room. In some embodiments, stat requests are received into a computer system from a dispatch manager.

At 302, one or more candidates are identified to perform a stat request. The candidates may be, for example, cleaners on a hospital staff.

At 304, for at least one of the candidates, a determination is made whether the candidate has a maximum number of stat requests. In some embodiments, the maximum number is a setting that can be set by a manager or administrator for all stat requests that come in. In one embodiment, the maximum number of stat requests is one stat request. In some embodiments, all of the candidate assignments are maintained a computer system. The determination of whether the candidate already has the maximum number of candidates may be made automatically by the computer system.

If the candidate does not have the maximum number of stat requests, the candidate is assigned to the stat request at 308. At 310, the candidate is paged with the assignment.

If the candidate already has the maximum number of stat requests, the stat request is not assigned to the designated cleaner, and a manager is notified that the candidate is not available to respond to the stat request at 312. At 314, a person other than the candidate is assigned to the stat request.

In some embodiments, candidate and alternate candidates are automatically identified by an EVS management system. In certain embodiments, some or all of the candidate cleaners are identified manually (for example, a manager making a selection on an electronic bed board).

In some embodiments, an escalation process is based on tasks not being initiated within a threshold amount of time. In one embodiment, upon receiving an indication of a dirty room/bed, a designated cleaner is assigned the task of cleaning the dirty room/bed (e.g., via a page to the designated cleaner). The system may monitor whether or not the task has been initiated within a threshold period of time (e.g., whether or not the designated cleaner has indicated starting cleaning of the room/bed within 30 min of the request). If it is determined that the task has been initiated within the threshold period of time, the usual workflow may continue (e.g., the system may wait for an indication of cleaning complete from the designated cleaner). If it is determined that the task has been not been initiated within the threshold period of time, the task may be escalated. For example, a manager may be notified of that the task has not been initiated within the threshold period of time. The task may be reassigned to another cleaner manually (e.g., by the manager) or automatically (e.g., by the system).

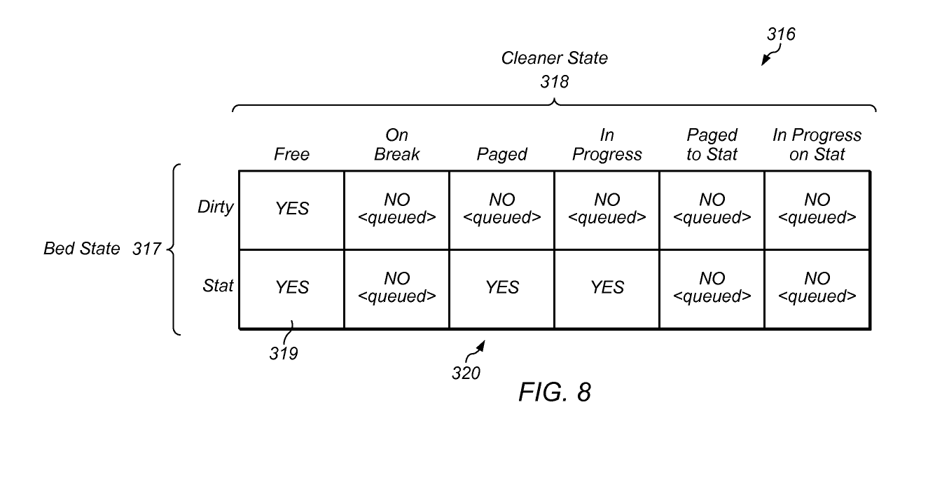

In some embodiments, cleaning requests are assigned based on a combination of a bed state and cleaner state. Assignments may be made by rules that implement values in a truth table. FIG. 8 illustrates one embodiment of a truth table for making bed cleaning assignments.

Cleaner truth table 316 includes values for combination of bed state 317 and cleaner state 318. In this example, bed states 317 include dirty or stat. Cleaner states 318 include Free, On Break, Paged, In Progress, Paged to Stat, and In Progress on Stat. In some embodiments, each page message may be sent to the truth table. The value for the combination of bed state 317 and cleaner state 318 may be used to determine how to manage a particular cleaning request. In some embodiments, the value for the combination of bed state 317 and cleaner state 318 determines whether a particular candidate cleaner is assigned a request. For example, if a cleaner has already been paged to a non-stat request, the truth table may be applied based on Paged column 320. If the bed state for the incoming request is stat, the candidate may be assigned the task and paged accordingly. If the bed state for the incoming request is stat, the candidate may be not be assigned the task and the request queued for further processing.

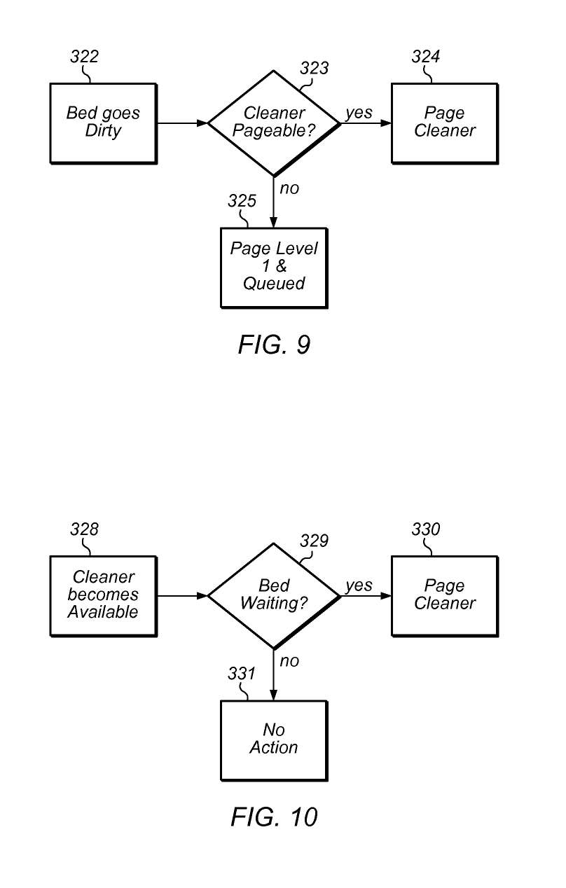

FIG. 9 is a flow diagram illustrating a cleaner page initiated based on a message that a bed has gone dirty. At 322, an indication is provided that a bed has gone dirty. A bed may go dirty, for example, at the time a patient is discharged from a room in a hospital. At 323, a determination is made whether a cleaner is pageable. In some embodiments, a determination of whether a cleaner is pageable for a particular assignment is based on a combination of a bed state and a cleaner state. For example, a cleaner state of Free may indicate that the cleaner is pageable. As another example, a cleaner state that the cleaner is In Progress on Stat may indicate that the cleaner is not available for any new cleaning assignments. As still another example, a cleaner state of In Progress on a non-stat cleaning request may indicate that the cleaner is pageable for new stat requests, but would not be pageable for non-stat requests.

If a determination made is that the cleaner is pageable, a page is sent to the cleaner at 324. The page may be sent automatically. In cases where the cleaner is indicated as not pageable (for example, due to the pager being In Progress cleaning another room), a cleaning request may be placed in a queue at 325. A Level 1 page may be issued. In some embodiments, a Level 1 page includes a notice to the cleaner or other cleaners that a room is dirty. The queued request may be sent to the cleaner upon completion of the non-stat request, or sent to another cleaner. In various embodiments, the determination of whether the cleaner is pageable, the page, and the queuing of the request are performed automatically by a computer system, such as an EVS management system.

In some embodiments, a page of a cleaner is triggered by a message that a cleaner has become available. FIG. 10 is a flow diagram illustrating paging based on a message that a cleaner has become available. At 328, an indication is provided that a cleaner has become available. A cleaner may become available, for example, when the cleaner starts the cleaner's shift, when the cleaner comes off break, or when the cleaner finished a previously assigned task. At 329, a determination is made whether a bed is waiting. A bed waiting may include either a standard bed dirty condition or a stat bed cleaning condition. If a bed is waiting, the available cleaner is paged at 330. If a bed is not waiting, no action is taken at 331.

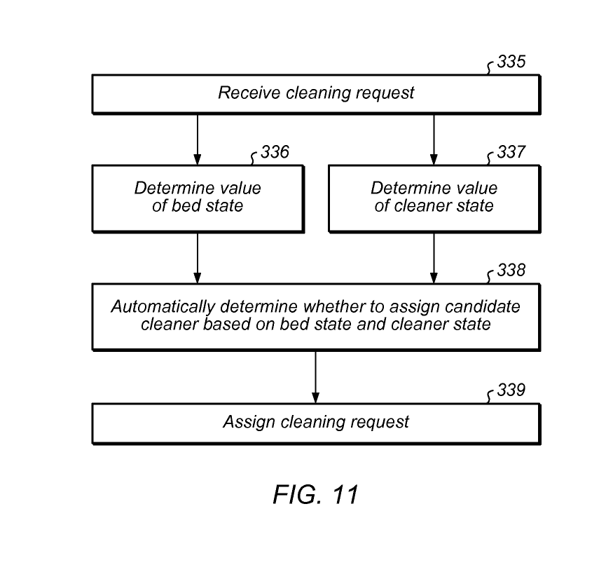

FIG. 11 illustrates one embodiment of making assignments based on a combination of values of bed state and cleaner state. At 335, a cleaning request is received. In some embodiments, a cleaning request is automatically generated. The request may be based, for example, on one or more messages or indicators from an admission/discharge/transfer system. At 336, a value of a bed state for the request is determined. In one embodiment, possible values of a bed state include: Dirty; and Stat.

At 337, a value of a cleaner state is determined for one or more candidate cleaners. In one embodiment, possible values of a cleaner state include Free, On Break; Paged; In Progress; Paged to Stat; and In Progress on Stat. At 338, a determination is made of whether to assign a candidate based, at least in part, on the values of the bed state for the request and the value of the cleaner state for one or more of the candidate cleaners. In some embodiments, a cleaner is selected based at least in part on the suitability of the cleaner. For example, the cleaner may be selected based on having experience with the tasks required in the request. At 339, a cleaner is assigned to the cleaning request.

In some embodiments, a cleaner is immediately paged for a request. In certain embodiments, a page may be delayed for a specific amount of time or until one or more conditions are met. For example, a cleaner may be paged to perform one task when the cleaner notifies the system that he or she has completed a previously assigned task.

In certain embodiments, if a cleaner finishes the cleaner's current room, or if a cleaner comes back from break, or if a cleaner receives a new assignment, then the queue of requests is examined to see if any of the queued messages requesting services can be delivered.

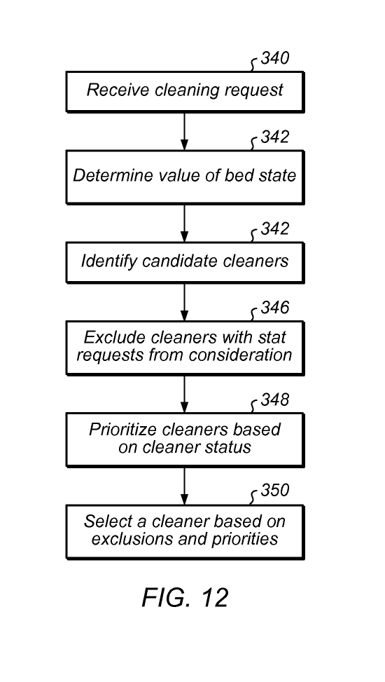

In some embodiments, cleaners with stat requests are excluded from consideration from other requests. FIG. 12 illustrates one embodiment of managing cleaning services that excludes cleaners with uncompleted stat requests. At 340, a cleaning request is received. At 342, a value is assessed for one or more beds in the request. In some embodiments, the possible values of bed state include a dirty state and a stat state.

At 344, a cleaner is selected for at least one of the requests from among two or more candidate cleaners. At 346, candidate cleaners are automatically excluded from consideration if the candidate cleaner has been paged for a prior stat request that is not yet completed; or is in progress on a prior stat request. At 348, candidate cleaners are prioritized based on cleaner status. In some embodiments, cleaners who are free have priority over cleaners who have been paged for a prior non-stat request that is not yet completed. Cleaners who are Free also have priority over cleaners who are in progress on a prior non-stat request. In some embodiments, cleaners are prioritized based on a suitability of the cleaners within the pool of candidates.

At 350, a cleaner is selected for the cleaning request. Selection of a cleaner may be based on exclusions for cleaners that have uncompleted stat requests, and on any prioritizations (such as prioritizations based on cleaner status).

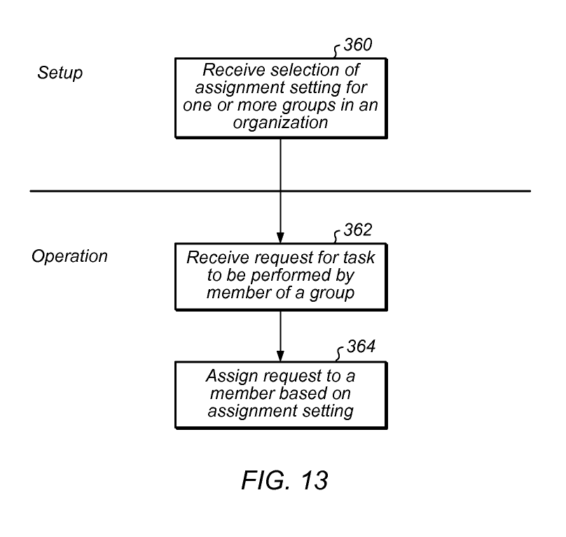

FIG. 13 illustrates one embodiment of assigning tasks based on a group assignment setting. At 360, a selection is received of an assignment setting for one or more groups or organizations. The setting may be established, for example, when a management system is initially placed into operation, during setup of the system, or at a later time. The assignment setting may be controlled by an administrator or by supervisory personnel at a health care facility. In one embodiment, a manager sets assignment codes for various units of the hospital to designate how tasks are to be assigned in each unit. In certain embodiments, assignment settings are set by each shift manager at the beginning of the shift.

In some embodiments, assignment settings for a group are defined by one or more codes. The codes may be entered in any manner, including keyboard entry, drop down menu, or any other means of entered a code choice. For example, a unit may be assigned “0” indicating that tasks are assigned manually (e.g., by the manager), a unit may be assigned “1” indicating that tasks are assigned to the designated cleaner for each particular task automatically (e.g., by the system), or a unit may be assigned “2” indicating that tasks are assigned to a cleaner automatically (e.g., by the system) based on availability (e.g., the cleaner that has been free for the longest time period).

Once the settings have been established, the system is operated to assign personnel to specific task requests, such as cleaning requests. At 362, a request is received for a task to be performed by a member of a group, such as the cleaning staff for a hospital unit. At 364, a member of the group is assigned based on the assignment setting. For example, in the example of codes given above relative to 360, if the code is set to 0, a manager may manually choose a cleaner for the assignment. If the code is set to 1, a cleaner may be assigned to the request automatically.

In certain embodiments, a cleaner is used in a reserve role. In one embodiment, a cleaner in a reserve role is assigned to a special zone having no beds. The cleaner may be available for the manager to manually dispatch assignments. A cleaner in a reserve role may not receive any automatic assignments from the system.

In some embodiments, a manager may put assign two or more cleaners to a reserve in a reserve role. In certain embodiments, all of the cleaners may be assigned to a reserve role. For example, on night shifts or other very low activity shifts, the manager may place all cleaners on reserve and dispatch all beds manually

In one embodiment, cleaning assignments are cleared at the end of each manager's shift. After the assignments are cleared, the manager for the subsequent shift may assign tasks to cleaning personnel.

### Misleading Message Detection

In various embodiments, a system automatically screens out, or adapts a response to, misleading messages. In one embodiment, messages generated by an ADT system are filtered to more accurately identify when bed cleaning requests are needed, thereby reducing, for example, premature requests for bed cleaning. A system may adapt a response in a manner that avoids wasting service resources (for example, a wasted trip to a room by cleaning personnel). In some embodiments, a system uses pattern matching to automatically detect misleading messages. For example, in cleaning management, the system may stop action from being taken to page cleaners or to change the states of bed unless a discharge or transfer that requires cleaning has actually occurred.

In various embodiments, messages coming into a system are assessed to determine whether they are misleading before personnel take action on the messages. In some embodiments, a service management system (such as an EVS management system) assesses messages fed from an ADT system. The system receiving the feed may determine whether the messages are misleading. An ADT message may be misleading, for example, if the message suggests that a patient is being discharged from a room when in fact the patient is not being discharged. For example, an HL7 message might indicate that a patient is being discharged, when in fact the patient is only undergoing a short procedure and will return to his or her room after the procedure.

In some embodiments, a system filters or adapts to messages indicating a “quick hop” scenario. In a “quick hop” scenario, a first message is received for the discharge of a first patient from a first bed, a second message is received for the admission of a second patient into the first bed previously occupied by the first patient, and due to a need to correct billing, misleading ADT messages are generated that indicate the second patient moving to another bed, the first patient being admitted into and discharged from the first bed, and the second patient being moved back into the first bed. The patients are not physically moved in response to the false messages, as the messages are merely entered into the system over a brief period of time (e.g., less than 5 min) to enable corrections in billing. If filtering of the messages is not applied, a cleaning request may be issued at each of the discharge messages, although cleaning may only be required when the patients are physically moved from the first bed (e.g., at the initial discharge of the first patient and the final discharge of the second patient). Similarly, in the context of intermediate transfers (e.g., transfers to Cardio Vascular Risk Assessments (CVRA) beds), a bed cleaning request may not be required as the patient is likely to return to their bed when an associated procedure is completed.

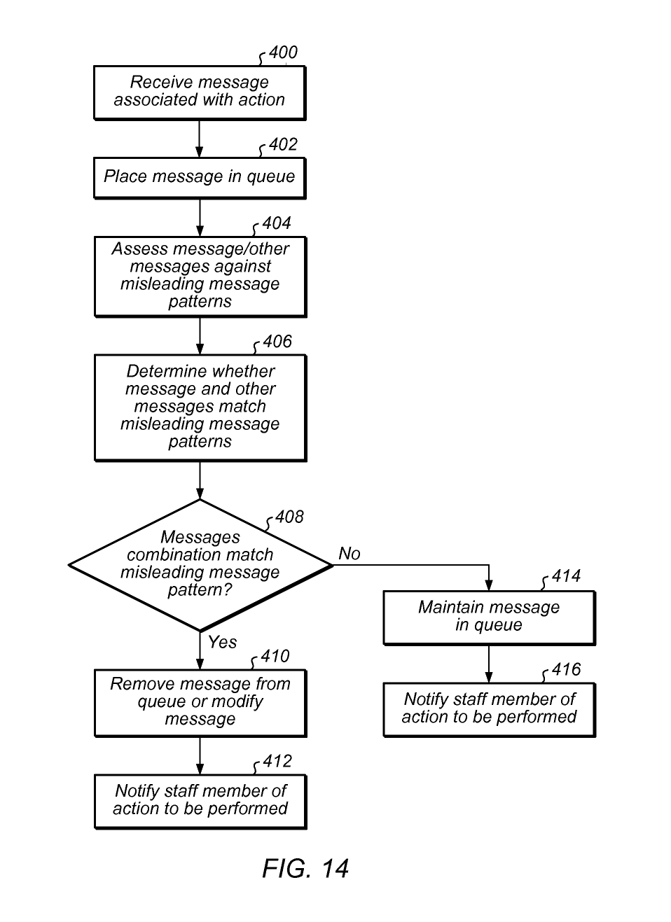

FIG. 14 illustrates one embodiment of managing services in a health care facility that includes detection of misleading messages. At 400, one or more messages associated with actions to be performed in the health care facility are received. In some embodiments, the messages are HL7 messages. The messages may be initiated, for example, from an ADT system. In some embodiments, the message may be a call message. The call message may specify an action to be performed, such as a bed cleaning. Messages received may be assessed automatically, manually, or a combination thereof.

At 402, one or more of the messages are placed in a queue. At 404, a queued message is assessed, in combination with one or more other messages, against one or more misleading message patterns stored in a memory. Each of the misleading message patterns may reflect a different set of messages or characteristics of messages indicating or suggesting that a message is misleading. Examples of characteristics of messages or sets of messages include: the type of message; the time between receiving one type of message and another of message; or the sequence of messages (for example, a discharge message followed quickly by an admission message). A message or set of messages may be assessed against any number of misleading message patterns. Assessing messages and message sets may be performed sequentially, in parallel, or a combination of both.

At 406, a determination is made of whether the combination of the message and the one or more other messages matches one or more of the misleading message patterns. A pattern match may indicate or suggest that the message is misleading. At 408, each message combination is assessed for a match with misleading message patterns. If one or more of the misleading message patterns indicates or suggests that the message is misleading, the item may be removed from the queue or the message modified at 410. At 412, a staff member to be assigned the task may be notified that a task is to be performed and provided with a description of the task.

If none of the misleading message patterns indicates or suggests that a message is misleading, the message in the queue may be maintained in the queue at 414. At 416, one or more staff member may be notified and assigned an action to be performed in association with the message.

In some embodiments, a cleaning request is issued based on the type of ADT message received. For example, where an ADT message to discharge a patient is received, a cleaning request may be issued, as the discharge message may indicate a high likelihood of the need for a bed cleaning request. Where an ADT message is received for cancelling a discharge of a patient, however, a cleaning request may not be issued, as the cancellation of a discharge may be indicative of a high likelihood that the message and other related messages are false and, thus, no cleaning request need be issued. For example, where a cancel discharge message is received—placing the first patient back into the first bed—the preceding and following messages relating to movements of the first and second patients may be ignored such that no cleaning request is issued.

In some embodiments, issuing a cleaning request may be based on the destination of a patient. For example, where a message is received indicating movement of a patient from a first bed to a second bed associated with brief procedure, such as CVRA bed, it may be recognized that the patient is likely to return to the first bed, and a cleaning request for the first bed may not be issued at that time. Notably, however, if a subsequent message is received indicating movement of the patient from the first or second bed to another bed (e.g., which is associated with an extended stay) a cleaning request for the first bed and the second bed may be issued at that time.

In some embodiments, a delay is provided in the analysis of a request such that related ADT messages can be considered for the determination of whether or not to issue a corresponding bed cleaning request. For example, in the context of “quick hops”, any billing corrections might be expected completed in a matter of 2-3 minutes. Accordingly, a 5 minute delay in processing messages and/or issuing a corresponding cleaning request may avoid issuing unneeded bed cleaning request based on false messages. For example, during the 5 minutes following the false message for the move of the second patient from the first to the second bed, a complete series of false messages request may be received, and processed to recognize no change in status (for example, the second patient remains in the first bed), such that no cleaning request is needed.

In some embodiments, managing services includes holding some or all of the messages received for a period of time. Messages on hold can be assessed, either alone or in combination with other messages, for characteristics that indicate or suggest that the messages are misleading messages. In some embodiments, held messages are assessed against misleading message patterns stored in the memory of a computer system. Held messages may be assessed automatically, manually, or a combination thereof.

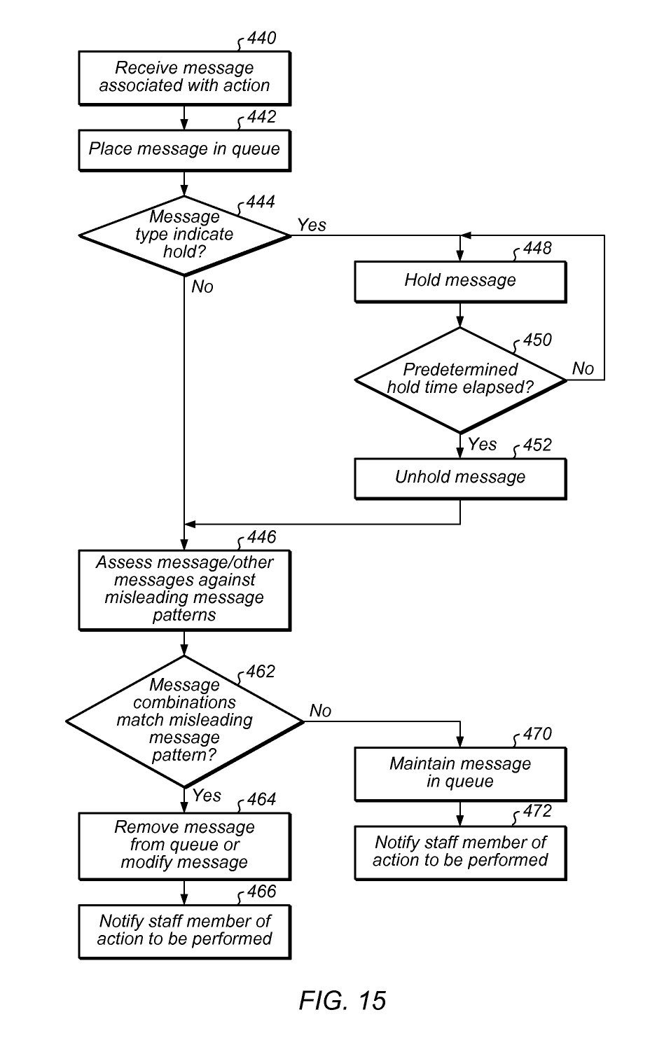

FIG. 15 illustrates one embodiment of managing services that includes holding and evaluating messages against patterns. At 440, one or more messages associated with one or more actions to be performed are received. At 442, messages are placed in a queue.

At 444, for one or more of the received messages, an assessment is made of based on one or more criteria to determine whether the message should be held. Criteria for determining whether a message should be held may include: characteristics of the message, such as the type of message (for example, admission, discharge, or transfer), characteristics of other messages, or the timing of the message relative to other messages.

One example of a message that may need to be held for assessment is a notification of a patient discharge. In many systems, a patient discharge message may immediately trigger a bed cleaning request. However, some patient discharge messages generated from an ADT system may be fictional messages that are later cancelled.

If the message is not of a type to be held, the message is processed in the queue at 446. If the received message should be held, a hold is applied to the message at 448. At 450, the message is tested as to whether a desired hold time has elapsed. The hold time for a message may be based on characteristics of the message, characteristics of other messages that appear to relate to the message, or the timing of the message relative to other messages. For example, the hold time for one type of message may be 30 seconds, while the hold time for another type of message may be 3 minutes. In some embodiments, the hold time can be modified by a user, such as a manager or administrator.

In certain embodiments, the hold time for a message is dynamically set. For example, the hold time for a message may change based on subsequent messages received from the system. For instance, a message may be released from hold based on the system receiving a subsequent message that validates the first message. As another example, the hold time for a message may be extended if a subsequent message raises the likelihood that the first message is misleading (increasing the suspicion level for the message).

Once the predetermined hold time has elapsed (or another condition has been met for releasing the hold), the message is unheld.

At 446, the unheld message is assessed, in combination with one or more other messages, against one or more misleading message patterns stored in a memory. At 462, a determination is made whether the combination of the unheld message and the one or more other messages matches one or more of the misleading message patterns. If one or more of the misleading message patterns indicates or suggests that the message is misleading, the message may be removed from the queue or modified at 464. At 466, a staff member (such as a bed cleaner) may be notified of the task and room location.

Modification of a message may include, for example, modifying timing of the task to be performed (such as delaying assignment or performance of the task) or the status of task (for example, stat or non-stat; complete or not complete). In certain embodiments, a staff member is notified of a cancellation or suspension of request for an action, such as a cleaning request previously assigned to the staff member.

If none of the misleading message patterns indicates or suggests that a message is misleading, the message may be maintained in the queue at 470. At 472, a staff member may be notified at least one staff member of at least one action to be performed in association with the message.

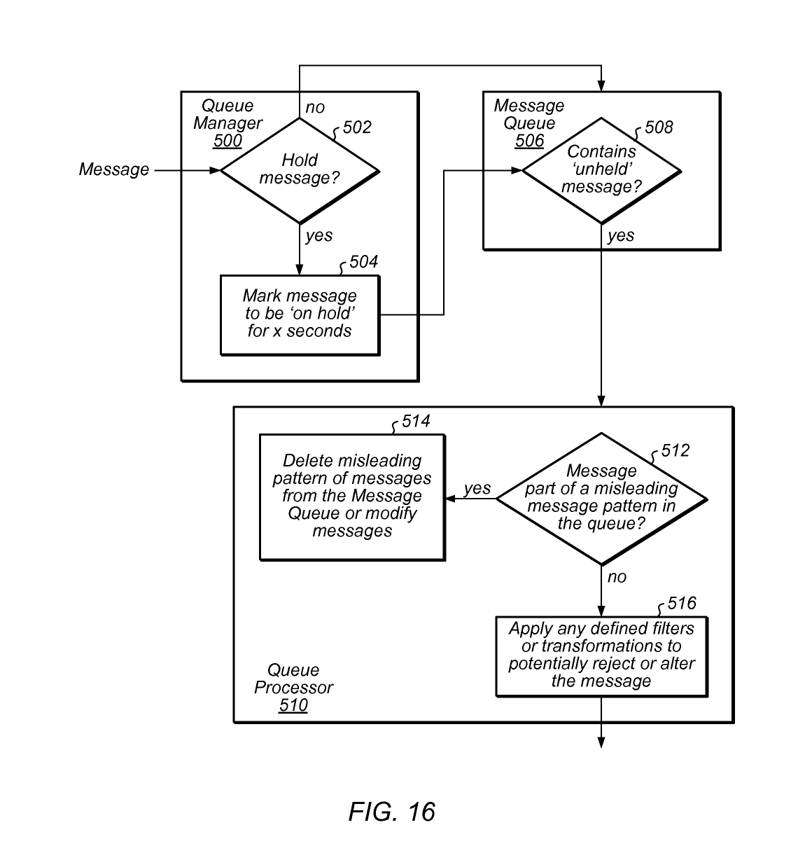

In some embodiments, a system implementing a delay pipeline detects and responds to misleading messages in a message feed used to trigger actions in a health care facility. FIG. 16 is a flow diagram illustrating one embodiment of a delay pipeline for responding to service requests.

A basic flow of a message through the delay pipeline may begin at message arrival at the Queue Manager program. In some embodiments, the message is an HL7 message. Queue Manager 500 may be responsible for determining if messages need to be put on hold. The messages may need to be put on hold, for example, if the messages may be later followed by a pattern of messages that indicates the initial message (and those following it that are part of the pattern) should be discarded. If it is determined a message should be put on hold at 502, Queue Manager 500 marks the message for a hold at 504 and indicates how long the message should be held in the Message Queue 506 before being processed. The message is then placed in Message Queue 506.

Each message is stored in Message Queue 506 until the message is no longer ‘on hold’ based on the elapsed time. At 508, when Message Queue 506 contains one or more unheld messages, Queue Processor 510 processes each message. The messages may be processed one at a time in the order they entered the queue in (for example, first in, first out). At 512, Queue Processor 510 looks at the current message and evaluates messages that are queued up after it to see if there is a match against one or more predefined misleading message patterns. If one of the misleading message patterns is identified, the system may remove from Queue Processor 510 some or all of the messages corresponding to the matched pattern at 514. The message may be deleted and discarded. If there are no matches with a misleading message pattern, the message may be processed by Queue Processor 510. Messages processed by Queue Processor 510 may have one or more custom filters or transformations applied as needed at 516 before continuing with processing of the message.

In some embodiments, Queue Manager 500 and Queue Processor 506 are part of a framework by which hold determination, misleading message detection, and filter/transformation logic are implemented. In one embodiment, hold determination, misleading message detection, and filter/transformation logic are implemented in a script written per-facility to define what logic is needed based on the facility the HL7 messages are coming from.

In some embodiments, when the amount of delay for messages has passed, the message is examined for the patterns and removed. If the message is not removed, then the message may be processed normally and sent to a bed board main system. A message may or may not trigger a notification to a staff member. For example, a bed moving from clean to occupied may not result in notification of any staff member, and may be considered a non-notifying event.

Different systems may generate different misleading messages and produce different misleading message patterns. In some embodiments, misleading message patterns are defined and stored for each of two or more different systems. For example, one set of misleading message patterns may be defined and installed on a system that will receive messages from an ADT system of type A, a different set of misleading message patterns may be defined and installed on a system that will receive messages from an ADT system of type B, and so on.

In some embodiments, a service management system, such as a bed cleaning management system, is used to manage services at two or more health care facilities. The system may receive messages from a message-generating system, such as ADT system, for each of the health care facilities. In some embodiments, the service management system is located in a cloud environment, such as described above relative to FIG. 2 and FIG. 3. For each different type of message generating system employed at the health care facilities, a different set of misleading message patterns may be installed into the memory of the service management system. During operation, as messages are received from each health care facility, the service management system may assess messages based on the set of patterns corresponding to the ADT system at the facility.

Computer systems may, in various embodiments, include components such as a CPU with an associated memory medium such as Compact Disc Read-Only Memory (CD-ROM). The memory medium may store program instructions for computer programs. The program instructions may be executable by the CPU. Computer systems may further include a display device such as monitor, an alphanumeric input device such as keyboard, and a directional input device such as mouse. Computer systems may be operable to execute the computer programs to implement computer-implemented systems and methods.

A computer system may allow access to users by way of any browser or operating system.

Computer systems may include a memory medium on which computer programs according to various embodiments may be stored. The term “memory medium” is intended to include an installation medium, e.g., Compact Disc Read Only Memories (CD-ROMs), a computer system memory such as Dynamic Random Access Memory (DRAM), Static Random Access Memory (SRAM), Extended Data Out Random Access Memory (EDO RAM), Double Data Rate Random Access Memory (DDR RAM), Rambus Random Access Memory (RAM), etc., or a non-volatile memory such as a magnetic media, e.g., a hard drive or optical storage. The memory medium may also include other types of memory or combinations thereof. In addition, the memory medium may be located in a first computer, which executes the programs or may be located in a second different computer, which connects to the first computer over a network. In the latter instance, the second computer may provide the program instructions to the first computer for execution. A computer system may take various forms such as a personal computer system, mainframe computer system, workstation, network appliance, Internet appliance, personal digital assistant (“PDA”), television system or other device. In general, the term “computer system” may refer to any device having a processor that executes instructions from a memory medium.

The memory medium may store a software program or programs operable to implement embodiments as described herein. The software program(s) may be implemented in various ways, including, but not limited to, procedure-based techniques, component-based techniques, and/or object-oriented techniques, among others. For example, the software programs may be implemented using ActiveX controls, C++ objects, JavaBeans, Microsoft Foundation Classes (MFC), browser-based applications (e.g., Java applets), traditional programs, or other technologies or methodologies, as desired. A CPU executing code and data from the memory medium may include a means for creating and executing the software program or programs according to the embodiments described herein.

Various embodiments may also include receiving or storing instructions and/or data implemented in accordance with the foregoing description upon a carrier medium. Suitable carrier media may include storage media or memory media such as magnetic or optical media, e.g., disk or CD-ROM, as well as signals such as electrical, electromagnetic, or digital signals, may be conveyed via a communication medium such as a network and/or a wireless link.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as embodiments. Elements and materials may be substituted for those illustrated and described herein, parts and processes may be reversed, and certain features of the invention may be utilized independently, all as would be apparent to one skilled in the art after having the benefit of this description of the invention. Methods may be implemented manually, in software, in hardware, or a combination thereof. The order of any method may be changed, and various elements may be added, reordered, combined, omitted, modified, etc. Changes may be made in the elements described herein without departing from the spirit and scope of the invention as described in the following claims.

1-43. (canceled)

44. A method of managing services in a health care facility, comprising:
receiving one or more messages associated with one or more actions to be performed in the hospital;
placing the one or more messages in a queue;
assessing a queued message, in combination with one or more other messages, against one or more misleading message patterns stored in a memory;
determining whether the combination of the message and the one or more other messages matches one or more of the misleading message patterns so as to indicate or suggest that the message is misleading; and
if one or more of the misleading message patterns indicates or suggests that the message is misleading, removing the message from the queue or modifying the message.

45. The method of claim 44, further comprising, if none of the misleading message patterns indicates or suggests that a message is misleading maintaining the message in the queue.

46. The method of claim 44, wherein at least one of the one or more messages comprises a call for cleaning services.

47. The method of claim 44, wherein the message is a bed cleaning request.

48. The method of claim 44, wherein the message is at least partially based on a fiction.

49. The method of claim 44, wherein the message is at least partially based on a fiction for administrative purposes.

50. The method of claim 44, wherein the message is at least partially based on a fiction for billing purposes.

51. The method of claim 44, wherein at least one of the misleading message patterns comprises one of the messages conflicting with at least one other message.

52. The method of claim 44, wherein at least one of the misleading message patterns comprises a later message cancelling or invalidating the message.

53. The method of claim 44, wherein at least one of the misleading message patterns comprises false discharge message.

54. The method of claim 44, wherein at least one of the misleading message patterns comprises a false indication that a patient is being moved from one bed to another.

55. The method of claim 44, wherein at least one of the misleading message patterns comprises a temporary absence of a patient from a bed.

56. The method of claim 44, wherein at least one of the misleading message patterns comprises a move from one bed to another for a specific procedure.

57. The method of claim 44, further comprising sending the message to an electronic bed board.

58. The method of claim 44, wherein the message is an admission/discharge/transfer message.

59. The method of claim 44, wherein the message is an HL7 message.

60. The method of claim 44, further comprising, before placing the message in the queue:
assessing whether or not at least one received message is a type of message to be held; and
if the received message is a type of message to be held:
applying a hold to the message;
unholding the message after a period of time has elapsed.

61. The method of claim 60, wherein the message is placed on hold for a specified time period.

62. The method of claim 61, further comprising determining the time period for holding the message, wherein the time period for the hold depends on the type of message.

63. The method of claim 60, wherein the message is assessed against the one or more misleading message patterns after the message is unheld.

64. The method of claim 44, further comprising, upon determining that the message is a misleading message, deleting the message from the queue.

65. The method of claim 44, further comprising, upon determining that the message is a misleading message, deleting the messages that match the misleading message pattern from the queue.

66. The method of claim 44, further comprising performing at least one transformation of the message after determining that the one or more misleading message patterns indicate that the message is not misleading.

67. The method of claim 44, further comprising performing at least one transformation of the message after determining that the one or more misleading message patterns indicate that the message is not misleading.

68. The method of claim 44, wherein messages are processed through the queue on a first-in, first-out basis.

69. A method of managing services, comprising:
receiving one or more messages associated with one or more actions to be performed;
placing the one or more messages in a queue;
assessing whether or not at least one received message is a type of message to be held;
if the received message is a type of message to be held, applying a hold to the message;
unholding the message after a period of time has elapsed;
assessing the unheld message, in combination with one or more other messages, against one or more misleading message patterns stored in a memory;
determining whether the combination of the unheld message and the one or more other messages matches one or more of the misleading message patterns so as to indicate or suggest that the message is misleading; and
if one or more of the misleading message patterns indicates or suggests that the message is misleading, removing the message from the queue or modifying the message.

70-83. (canceled)

84. A system for managing cleaning requests, comprising:
a processor;
a memory coupled to the processor and configured to store program instructions executable by the processor to implement:
receiving one or more messages associated with one or more actions to be performed in the hospital;
placing the one or more messages in a queue;
assessing a queued message, in combination with one or more other messages, against one or more misleading message patterns stored in a memory;
determining whether the combination of the message and the one or more other messages matches one or more of the misleading message patterns so as to indicate or suggest that the message is misleading; and
if one or more of the misleading message patterns indicates or suggests that the message is misleading, removing the message from the queue or modifying the message.

85-87. (canceled)
